(19) United States
(12) Patent Application Publication
(10) Pub. No.: US 2020/0187542 A1
(43) Pub. Date: Jun. 18, 2020

(54) GENDER SPECIFIC SYNTHETIC NUTRITIONAL COMPOSITIONS AND NUTRITIONAL SYSTEMS COMPRISING THEM

(71) Applicant: SOCIETE DES PRODUITS NESTLE S.A., Vevey (CH)

(72) Inventors: Sagar Thakkar, Brent (CH); Francesca Giuffrida, Mezieres (CH); Carlos Antonio De Castro, Geneva (CH); Frederic Destaillats, Servion (CH)

(21) Appl. No.: 16/641,314

(22) PCT Filed: Aug. 15, 2018
(86) PCT No.: PCT/EP2018/072139

§ 371 Date: Feb. 24, 2020

(30) Foreign Application Priority Data
Aug. 22, 2017 (EP) 17187280.7

(51) Int. Cl. A23L 33/15; A23L 33/00; A61K 31/355

(52) U.S. Cl. CPC A23L 33/15; A23V 2002/00; A61K 31/355; A23L 33/40

(57) ABSTRACT

Gender specific synthetic nutritional compositions comprising gamma tocopherol in concentrations reflecting those found in human milk produced by mothers of infants of the corresponding gender at the corresponding stage of lactation, and nutritional systems comprising them.

### TECHNICAL FIELD

The invention relates to gender specific synthetic nutritional compositions, to nutritional systems comprising them, and to their use to provide an optimised amount of gamma tocopherol and/or one or more health benefit to an infant.

### BACKGROUND OF THE INVENTION

Even though breastfeeding is optimal for infants, the existence of certain conditions may mean that it is contraindicated (AAP, 2012; Lawrence, 2013). In such cases, where the sole source of nutrition is not available to infants, alternative strategies to feed them have to be devised. Feeding infants with synthetic nutritional compositions e.g. Infant formula is one such strategy.

The compositions of the aforementioned synthetic nutritional compositions e.g. infant formulas, aim to replicate those of human milk (hereinafter HM). However, replicating HM is not a simple task. HM not only contains numerous components, its composition is extremely dynamic and these dynamic changes remain largely unexplored and uncharacterized.

The inventors have now surprisingly found that the concentration of gamma tocopherol in HM may differ depending on the stage of lactation and the gender of a mother's infant. Because such age and gender differences in the gamma tocopherol concentration of HM have never previously been identified, these differences are not reflected in the compositions of synthetic nutritional compositions available for infants today. Given that HM is considered the gold standard with respect to infant nutrition, there remains a need for synthetic nutritional compositions tailored for infants of specific ages and genders which better reflect these identified differences.

### SUMMARY OF THE INVENTION

The invention is set out in the claims. The inventors have developed gender specific synthetic nutritional compositions for infants comprising gamma tocopherol in concentrations that reflect the concentration of gamma tocopherol found in HM produced for an infant of the same age/corresponding lactation stage and gender.

Said gender specific synthetic nutritional compositions may for example be an infant formula or a composition for an infant that is intended to be added to or diluted with human milk.

The gender specific synthetic nutritional compositions of the invention can be prepared from a gender neutral synthetic nutritional composition by measuring out an appropriate amount of said gender neutral synthetic nutritional composition and mixing it with an additive and/or diluent e.g. gamma tocopherol and/or water.

The gender specific synthetic nutritional compositions of the invention may be included in a nutritional system. Said nutritional system may comprise a gender specific synthetic nutritional composition for a female infant and/or a gender specific composition for a male infant of the same age. A gender specific synthetic nutritional composition for a male infant may comprise more gamma tocopherol than a gender specific synthetic nutritional composition for a female infant of the same age, and said gender specific synthetic nutritional compositions may be for infants of an age selected from the group consisting of: up to 2 months of age, and 4 months of age or older. Alternatively or in addition, the nutritional system may comprise a gender specific synthetic nutritional composition for a male infant that may comprise less gamma tocopherol than a gender specific synthetic nutritional composition for a female infant of the same age, and said gender specific synthetic nutritional compositions may be for infants of 2 to 4 months of age or older.

The gamma tocopherol concentration of a gender specific synthetic nutritional composition of the invention reflects the gamma tocopherol concentration found in HM produced for an infant of the same gender and age (at a corresponding lactation stage). Because HM is considered optimal with respect to infant nutrition, a gender specific synthetic nutritional composition of the invention, and therefore a nutritional system comprising same, may provide an optimized amount of gamma tocopherol to an infant, for example an infant having the same gender as the gender to whom the synthetic nutritional composition is directed, and may be used to ensure optimum gamma tocopherol levels and thereby to optimize antioxidant capacity as well as immunomodulation.

### DETAILED DESCRIPTION

The inventors performed a longitudinal study evaluating the nutrient composition of HM collected from mothers at various stages of lactation (30 days (1 month), 60 days (2 months), and 120 days (4 months) postpartum). Surprisingly the results of this study indicated that the concentration of gamma tocopherol found in HM can differ depending on the stage of lactation and/or the gender of a mother's infant. In particular this study indicated that up to 2 months postpartum and 4 months postpartum and later, the concentration of gamma tocopherol may be higher in HM produced by mothers to boys than in HM produced by mothers to girls. The study further indicated that at 2 to 4 months postpartum the concentration of gamma tocopherol may be lower in HM produced by mothers to boys than in HM produced by mothers to girls. Details of the study, analysis techniques and results are given in example 1.

Based on the findings of the study, the inventors have designed gender specific synthetic nutritional compositions that comprise gamma tocopherol in a concentration that reflects the gamma tocopherol concentration found in HM produced for an infant of the same gender at the corresponding stage of lactation.

The term “gender specific synthetic nutritional composition” as used herein refers to any synthetic nutritional composition, intended to be consumed by an infant that is specifically adapted to the nutritional needs of either a female or male enfant. Non limiting examples of gender specific synthetic nutritional compositions for infants from birth to 4 months include; infant formulae, and a composition for infants that is intended to be added or diluted with HM e.g. HM fortifier. Non limiting examples of gender specific synthetic nutritional compositions for infants from 4 months to 12 months include infant formulae, a composition for infants that is intended to be added or diluted with HM e.g. HM fortifier, or food stuffs intended for consumption by infants either alone or in combination with HM e.g. complementary foods.

The term “infant formula” or “infant formulae” as used herein refers to a nutritional formulation (either in the form of a liquid or in the form of a dry powder that may be reconstituted to form a liquid infant formula upon addition of water) that provides complete nutrition for an infant and is suitable to feed an infant, and which meets the US or EU standards for infant formula. Such formulae are well-known in the art.

The term “infant” as used herein refers to a human infant of 12 months of age or less.

In an aspect of the present invention there is provided a gender specific synthetic nutritional composition tailored for an infant comprising gamma tocopherol in a concentration reflecting the concentration found in HM produced for an infant of the same gender at the corresponding lactation stage e.g. up to 2 months, 2 to 4 months, 4 months and later.

In an embodiment the gender specific synthetic nutritional composition is a male gender specific synthetic nutritional composition for an infant of up to 2 months of age and comprises gamma tocopherol in a concentration selected from the group consisting of: 1.5 to 7.6, 2.5 to 4.8, 4 to 4.2 μg/mL.

In an embodiment the gender specific synthetic nutritional composition is a female gender specific synthetic nutritional composition for an infant of up to 2 months of age and comprises gamma tocopherol in a concentration selected from the group consisting of: 1.1 to 8, 2.6 to 4.8, 3.2 to 3.8 μg/mL.

Non limiting examples of ages up to 2 months of age include; up to 2 weeks, up to 1 month, 1 month, and 1 month up to 2 months of age.

In an embodiment the gender specific synthetic nutritional composition is a male gender specific synthetic nutritional composition for an infant of 2 months up to 4 months of age and comprises gamma tocopherol in a concentration selected from the group consisting of: 0.5 to 5.5, 1.5 to 3.9, 2.8 to 3 μg/mL.

In an embodiment the gender specific synthetic nutritional composition is a female gender specific synthetic nutritional composition for an infant of 2 months up to 4 months of age and comprises gamma tocopherol in a concentration selected from the group consisting of: 0.5 to 8, 1.3 to 4.2, 3 to 3.2 μg/mL.

Non limiting examples of ages 2 months up to 4 months include; 2 months, 2 months up to 3 months, 3 months, 3 months up to 4 months.

In an embodiment the gender specific synthetic nutritional composition is a male gender specific synthetic nutritional composition for an infant of 4 months of age and older and comprises gamma tocopherol in a concentration selected from the group consisting of: 0.6 to 7.7, 2.3 to 5, 2.9 to 3.7 μg/mL.

In an embodiment the gender specific synthetic nutritional composition is a female gender specific synthetic nutritional composition for an infant of 4 months of age and older and comprises gamma tocopherol in a concentration selected from the group consisting of: 0.4 to 4.7, 1.6 to 3.2, 2.4 to 2.8 μg/mL.

Non limiting examples of an age 4 months of age and older include; 4, 5, 6, 7, 8, 9, 10, 11, and 12 months of age, 4 to 6 months of age, 4 to 12 months of age, 6 to 12 months of age, 6 to 9 months of age, and 9 to 12 months of age.

The gamma tocopherol concentration of the gender specific synthetic nutritional compositions defined herein is expressed in μg/mL. This may refer to the gamma tocopherol concentration of a reconstituted gender specific synthetic nutritional composition.

The term gamma tocopherol as used herein refers to total gamma tocopherol including gamma tocopherol esters.

The gamma tocopherol concentration of a composition can be measured by methods well known in the art. In particular the gamma tocopherol concentration can be measured using liquid-liquid extraction using organic solvents followed by separation on liquid chromatography using a calibration curve and detection by UV/DAD detectors.

Any form of gamma tocopherol suitable for administration to an infant to whom the gender specific synthetic nutritional composition is directed may be comprised within in the gender specific synthetic nutritional compositions of the invention. The gamma tocopherol may for example be added in the ester form e.g. in the form of racemic tocopherol acetate.

The gamma tocopherol, in any form it is used, may stem from natural sources, in particular it may stem from animal or plant sources such as vegetable oil, in particularly it will stem from rosehip, corn and/or soya bean oil.

The gender specific synthetic nutritional compositions of the invention can also comprise any other ingredients or excipients known to be employed in the type of gender specific synthetic nutritional composition in question e.g. infant formula.

Non limiting examples of such ingredients include: proteins, amino acids, carbohydrates, oligosaccharides, lipids, prebiotics or probiotics, essential fatty acids, nucleotides, nucleosides, other vitamins, minerals and other micronutrients.

Non limiting examples of proteins include: casein, alpha-lactalbumin, whey, soy protein, rice protein, corn protein, oat protein, barley protein, wheat protein, rye protein, pea protein, egg protein, sunflower seed protein, potato protein, fish protein, meat protein, lactoferrin, serum albumin, immunoglobins, and combinations thereof.

Non limiting examples of amino acids include leucine, threonine, tyrosine, Isoleucine, arginine, alanine, histidine, isoleucine, proline, valine, cysteine, glutamine, glutamic acid, glycine, serine, arginine, lysine, methionine, phenylalanine, tryptophane, asparagine, aspartic acid, and combinations thereof.

Non limiting examples of carbohydrates include lactose, saccharose, maltodexirin, starch, and combinations thereof.

Non limiting examples of lipids include: palm olein, high oleic sunflower oil, high oleic safflower oil, canola oil, fish oil, coconut oil, bovine milk fat, and combinations thereof.

Non limiting examples of essential fatty acids include: linoleic acid (LA), α-linolenic acid (ALA) and polyunsaturated fatty acids (PUFAs). The gender specific synthetic nutritional compositions of the invention may further contain gangliosides monosialoganglioside-3 (GM3) and disialogangliosides 3 (GD3), phospholipids such as sphingomyelin, phospholipids phosphatidylcholine, phosphatidylethanolamine, phosphatidylinositol, phosphatidylserine, and combinations thereof.

None limiting examples of prebiotics include: oligosaccharides optionally containing fructose, galactose, mannose; dietary fibers, in particular soluble fibers, soy fibers; inulin; and combinations thereof. Preferred prebiotics are fructo-oligosaccharides (FOS), galacto-oligosaccharides (GOS), isomalto-oligosaccharides (IMO), xylo-oligosaccharides (XOS), arabino-xylo oligosaccharides (AXOS), mannan-oligosaccharides (MOS), oligosaccharides of soy, glycosylsucrose (GS), lactosucrose (LS), lactulose (LA), palatinose-oligosaccharides (PAO), malto-oligosaccharides, gums and/or hydrolysates thereof, pectins and/or hydrolysates thereof, and combinations of the foregoing.

Further examples of oligosaccharide are described in Wrodnigg, T. M.; Stutz, A. E. (1999) Angew. Chem. Int. Ed. 38:827-828 and in WO 2012/069416 which is incorporated herein by reference.

Non limiting examples of probiotics include: Bifidobacterium, Lactobacillus, Lactococcus, Enterococcus, Streptococcus, Kluyveromyces, Saccharoymces, Candida, in particular selected from the group consisting of Bifidobacterium longum, Bifidobacterium lactis, Bifidobacterium animalis, Bifidobacterium breve, Bifidobacterium infantis, Bifidobacterium adolescentis, Lactobacillus acidophilus, Lactobacillus casei, Lactobacillus paracasei, Lactobacillus salivarius, Lactobacillus lactis, Lactobacillus rhamnosus, Lactobacillus johnsonii, Lactobacillus plantarum, Lactobacillus salivarius, Lactococcus lactis, Enterococcus faecium, Saccharomyces cerevisiae, Saccharomyces boulardii or mixtures thereof, preferably selected from the group consisting of Bifidobacterium longum NCC3001 (ATCC BAA-999), Bifidobacterium longum NCC2705 (CNCM I-2618), Bifidobacterium longum NCC490 (CNCM 1-2170), Bifidobacterium lactis NCC2818 (CNCM 1-3446), Bifidobacterium breve strain A, Lactobacillus paracasei NCC2461 (CNCM 1-2116), Lactobacillus johnsonii NCC533 (CNCM 1-1225), Lactobacillus rhamnosus GG (ATCC53103), Lactobacillus rhamnosus NCC4007 (CGMCC 1.3724), Enterococcus faecium SF 68 (NCC2768; NCIMB10415), and combinations thereof.

Non limiting examples of Nucleotides include: cytidine monophosphate (CMP), uridine monophosphate (UMP), adenosine monophosphate (AMP), guanosine monophosphate (GMP), and combinations thereof.

Non limiting examples of vitamins and minerals include: vitamin A, vitamin B1, vitamin B2, vitamin B6, vitamin B12, vitamin K, vitamin C, vitamin D, folic acid, inositol, niacin, biotin, pantothenic acid, choline, calcium, phosphorous, iodine, iron, magnesium, copper, zinc, manganese, chloride, potassium, sodium, selenium, chromium, molybdenum, taurine, L-carnitine, and combinations thereof. Minerals are usually added in salt form.

Other suitable and desirable ingredients of synthetic nutritional compositions, that may be employed in the gender specific synthetic nutritional compositions of the invention are described in guidelines issued by the Codex Alimentarius with respect to the type of synthetic nutritional composition in question e.g. Infant formula, HM fortifier, follow on formula, or food stuffs intended for consumption by infants e.g. complementary foods.

The gender specific synthetic nutritional compositions of the invention may be prepared by methods well known in the art for preparing the type of gender specific synthetic nutritional composition in question e.g. infant formulae, follow on formulae, a composition for infants that is intended to be added or diluted with HM e.g. HM fortifier, or food stuffs intended for consumption by infants either alone or in combination with HM e.g. complementary foods.

An exemplary method for preparing a gender specific powdered infant formula is as follows. A protein source, carbohydrate source, and fat source may be blended together in appropriate proportions. Gamma tocopherol may be added or may be inherently comprised within a protein, carbohydrate and/or fat source. Emulsifiers maybe included in the blend. Vitamins and minerals may be added at this point but are usually added later to avoid thermal degradation. Any lipophilic vitamins, emulsifiers and the like may be dissolved into the fat source prior to blending. Water, preferably water which has been subjected to reverse osmosis, may then be mixed in to form a liquid mixture.

The liquid mixture may then be thermally treated to reduce bacterial loads. For example, the liquid mixture may be rapidly heated to a temperature in the range of about 80° C. to about 110° C. for about 5 seconds to about 5 minutes. This may be carried out by steam injection or by heat exchanger; for example a plate heat exchanger.

The liquid mixture may then be cooled to about 60° C. to about 85° C.; for example by flash cooling. The liquid mixture may then be homogenised; for example in two stages at about 7 MPa to about 40 MPa in the first stage and about 2 MPa to about 14 MPa in the second stage.

The homogenised mixture may then be further cooled to add any heat sensitive components such as vitamins and minerals. The pH and solids content of the homogenised mixture is conveniently standardised at this point.

The homogenised mixture can be transferred to a suitable drying apparatus such as a spray drier or freeze drier and converted to powder. The powder should have a moisture content of less than about 3% by weight.

If it is desired probiotic(s) can be added, they may be cultured according to any suitable method and prepared for addition to the infant formula by freeze-drying or spray-drying for example. Alternatively, bacterial preparations can be bought from specialist suppliers such as Christian Hansen and Morinaga already prepared in a suitable form for addition to food products such as infant formula. Such bacterial preparations may be added to the gender specific powdered infant formula by dry mixing.

The gender specific synthetic nutritional compositions of the invention may also be prepared from a gender neutral synthetic nutritional composition in a method comprising; measuring out an appropriate amount of said gender neutral synthetic nutritional composition and mixing it with an additive and/or a diluent e.g. gamma tocopherol and/or water so as to arrive at a gender specific synthetic nutritional composition in accordance with the invention.

The additive may be a gender specific additive comprising gamma tocopherol in a particular concentration so that when mixed with the gender neutral synthetic nutritional composition, and optionally a diluent, the resulting mixture is a gender specific synthetic nutritional composition in accordance with the invention.

The gender neutral synthetic nutritional composition can be prepared by methods well known in the art for the type of composition in question e.g. as laid out above for infant formula.

The term “gender neutral” as used herein is synonymous with unisex.

One or more of the gender specific synthetic nutritional compositions of the invention can be included in a nutritional system.

The term “nutritional system” as used herein refers to a collection of more than one synthetic nutritional composition advertised or sold as part of the same product range e.g. a collection of infant formulas sold under the same brand and adapted/tailored to the nutritional needs of infants of differing ages and/or genders and/or delivered by different methods e.g. C-section. The synthetic nutritional compositions making up the nutritional system may be packaged individually e.g. in capsules or boxes. Said packages can be sold individually, grouped together e.g. wrapped by plastic film or combined in a box, or in a combination of these two ways. The nutritional system may also comprise synthetic nutritional compositions for children older than 12 months.

In a further aspect of the present invention there is provided a nutritional system comprising a gender specific synthetic nutritional composition of the invention.

In an embodiment the nutritional system comprises a gender specific synthetic nutritional composition for a male infant and a gender specific synthetic nutritional composition for a female infant wherein said male and female gender specific synthetic nutritional compositions are for infants of the same age and wherein the concentration of gamma tocopherol in said gender specific synthetic nutritional composition for a male infant differs from that in said gender specific synthetic nutritional composition for a female infant.

In a more specific embodiment the nutritional system comprises a gender specific synthetic nutritional composition for a male infant of up to 2 months of age, and a gender specific synthetic nutritional composition for a female infant of up to 2 months of age wherein, the concentration of gamma tocopherol in said male gender specific synthetic nutritional composition is higher than the gamma tocopherol concentration of said female gender specific synthetic nutritional composition.

The concentration of gamma tocopherol in said male gender synthetic nutritional compositions may be higher by any amount.

Said male gender specific synthetic nutritional composition may comprise for example 0.1 to 6.5, 0.3 to 4, 0.1 to 1 μg/mL more gamma tocopherol than the female gender specific synthetic nutritional composition.

In another specific embodiment the nutritional system comprises a gender specific synthetic nutritional composition for a male infant of from 4 months of age, and a gender specific synthetic nutritional composition for a female infant of from 4 months of age wherein, the concentration of gamma tocopherol in said male gender specific synthetic nutritional composition is higher than the gamma tocopherol concentration of said female gender specific synthetic nutritional composition.

The concentration of gamma tocopherol in said male gender synthetic nutritional compositions may be higher by any amount.

Said male gender specific synthetic nutritional composition may comprise for example 0.1 to 7.5, 1 to 5.5, 0.1 to 1.2 μg/mL more gamma tocopherol than the female gender specific synthetic nutritional composition.

In yet another specific embodiment the nutritional system comprises a gender specific synthetic nutritional composition for a male infant of 2 months to 4 months of age, and a gender specific synthetic nutritional composition for a female infant of 2 months to 4 months of age wherein, the concentration of gamma tocopherol in said male gender specific synthetic nutritional composition is lower than the gamma tocopherol concentration of said female gender specific synthetic nutritional composition.

The concentration of gamma tocopherol in said female gender synthetic nutritional compositions may be higher by any amount.

Said female gender specific synthetic nutritional composition may comprise for example 0.1 to 7, 0.3 to 5, 1.5 to 4, 0.1 to 0.3 μg/mL more gamma tocopherol than the male gender specific synthetic nutritional composition.

Statistical analysis of the results of the longitudinal study described herein indicated that gender differences in the gamma tocopherol concentration of HM at 4 months (120 days) postpartum may be significant.

Accordingly, it may be that the nutritional systems of the invention only comprise a gender specific synthetic nutritional composition of the invention for an infant 4 months of age and older e.g. 4, 5, 6, 7, 8, 9, 10, 11, 12 months of age or any range therein. Such a nutritional system may comprises gender neutral synthetic nutritional compositions for infants up to 4 months of age, more particularly for infants up to 2 months of age.

The nutritional system may further comprise nutritional compositions for children older than 12 months.

Gender specific synthetic nutritional compositions according to the invention are particularly suitable for use in a method of preparing single servings of infant formula using capsules, each capsule of which contains a unit dose of a synthetic nutritional composition e.g. a gender specific synthetic nutritional composition in a concentrated form, and which is equipped with opening means contained within the capsule to permit draining of the reconstituted synthetic nutritional composition directly from the capsule into a receiving vessel such as a baby bottle. Such a method is described in WO2006/077259.

The different synthetic nutritional compositions, including synthetic nutritional compositions tailored for an infant of a specific age may be packed into individual capsules and presented to the consumer in multipacks containing a sufficient number of capsules to meet the requirements of an infant of a particular age or age range, for one week for example. Suitable capsule constructions are disclosed in WO2003/059778.

The different synthetic nutritional compositions, including gender specific and gender neutral synthetic nutritional compositions, which may be comprised within a nutrition system, may be packed into individual capsules and presented to the consumer in multipacks containing a sufficient number of capsules to meet the requirements of an infant of a particular age or range for one week for example. Suitable capsule constructions are disclosed in WO2003/059778.

The capsules can contain the synthetic nutritional compositions, (gender specific and gender neutral) in the form of powders or concentrated liquids in both cases for reconstitution by an appropriate amount of water. Both the composition and the quantity of infant formula in the capsules may vary according to the gender and/or age of the infant. If necessary, different sizes of capsules may be provided for the preparation of infant formulas for infants of different genders and/or ages.

Because HM is the gold standard when it comes to infant nutrition, and because the gamma tocopherol concentration of the gender specific synthetic nutritional compositions of the invention better reflect the gamma tocopherol concentration found in HM at the corresponding lactation stage for mothers of infants of the corresponding gender, they, and the nutritional systems comprising them, may be used to provide an optimum amount of gamma tocopherol to an infant and to ensure optimum gamma tocopherol levels and to prevent conditions associated with non-optimal gamma tocopherol levels and to optimise antioxidant capacity and immunomodulation.

Long term effects may only be evident in months or years e.g. 6 months, 9 months, 12 months, 5 years, 10 years, or 20 years.

In another aspect of the present invention there is provided a gender specific synthetic nutritional composition of the invention for use to prevent and/or treat a condition associated with non-optimal gamma tocopherol levels.

Non limiting examples of conditions associated with non-optimal gamma tocopherol levels include hypovitaminosis, abetalipoproteinemia, and fat malabsorption.

The gender specific synthetic nutritional compositions of the invention may provide an optimum amount of gamma tocopherol to an infant, in particular an infant up to 2 months of age, 2 to 4 months of age, or 4 months of age or older.

The nutritional system may for example provide an optimum amount of gamma tocopherol to an infant, in particular for an infant up to 12, 11, 10, 9, 8, 7, 6, 5, 4, 3, 2, 1 months of age and/or up to 2 weeks of age.

In another aspect of the present invention there is provided a method for providing an optimum amount of gamma tocopherol to an infant comprising:
a) Optionally preparing a gender specific synthetic nutritional composition of the invention from a gender-neutral synthetic nutritional composition;
b) Feeding a gender specific synthetic nutritional composition according to the invention to an infant, in particular an infant of the corresponding gender and age, more particularly an infant of up to 2 months of age, 2 to 4 months of age, or 4 months of age or older.

As stated herein. The gender specific synthetic nutritional compositions may be prepared from gender neutral synthetic nutritional compositions. Accordingly, in another aspect of the present invention there is provided a kit for providing an optimized amount of gamma tocopherol to an infant, in particular an infant up to 2 months of age, 2 to 4 months of age, or 4 months of age or older, the kit comprising:
a) A gender neutral synthetic nutritional composition
b) A label indicating dosage requirements for an infant so as to arrive at a gender specific nutritional composition in accordance with the invention.

The dosage requirements may be with respect to the quantity of the gender neutral synthetic nutritional employed and/or consumption frequency e.g. 4 times per day.

It should be appreciated that all features of the present invention disclosed herein can be freely combined and that variations and modifications may be made without departing from the scope of the invention as defined in the claims. Furthermore, where known equivalents exist to specific features, such equivalents are incorporated as if specifically referred to in this specification.

There now follows a series of non-limiting examples that serve to illustrate the invention.

### EXAMPLES

### Example 1

Longitudinal Clinical Trial:

The present inventors designed a longitudinal clinical trial with 50 lactating mothers with milk sampling at 30 (visit 1), 60 (visit 2) and 120 (visit 3) days post-partum. The milk samples were quantitatively analyzed for gamma tocopherol.

Human Milk Collection:

The protocol and collection of human milk was reviewed and approved by the local ethical committee of Singapore. The study took place at National University of Singapore. Volunteer mothers of term infants, who were apparently healthy and non-smokers (n=50; 31.1±3.1-year old) provided breast milk samples (approximately 30 mL). Samples were collected after full expression from one breast using a milk pump, while the baby was fed on the other breast. All efforts were made to collect complete feed that included fore-milk, mid-milk and hind-milk as a representation of one feed, to avoid within feed variation of lipid content. Approximately 30 mL aliquot was separated in a conical polypropylene tube for this study and the rest was fed to the infant. Samples collected for research were stored at −80° C. until analyses. Data collection points were 30 days (1 month), 60 days (2 months) and 120 days (4 months) postpartum.

Measurement of the Gamma Tocopherol Concentrations in Samples:

The gamma tocopherol concentration of each sample was measured by liquid-liquid extraction using organic solvents followed by separation on liquid chromatography using a calibration curve and detection by UV/DAD detectors. The gamma tocopherol standard curve is constructed by plotting the response (Area) versus the concentration using weighted linear regression model.

The resulting calibration curve equation is as follows:

y=a·x+b

Where y is the calibration response, x is the know concentration of the gamma tocopherol, a is the slope and b is the intercept.

The mass fraction of the gamma tocopherol in the sample, in mg/100 mL sample, is calculated using the following equation:

$$\frac{C \cdot 100}{m}$$

where:
C=concentration of the gamma tocopherol in the sample test solution obtained using the calibration curve, in μg/mL;
100=conversion factor to express the final result in mg/100 mL sample;
m=mass of the test portion, in mL;

The results of the analysis of the HM, with respect to gamma tocopherol concentration are shown in tables 1a and 1 b.

TABLE 1a

| | Gamma tocopherol Concentration μg/mL | | | | | |
|---|---|---|---|---|---|---|
| | Female | | | | | |
| Stage | Min | Median | Max | SD | QT1 | QT3 |
| 30 days | 1.174604 | 3.20065474 | 8.18180286 | 1.90365767995704 | 2.620765425 | 4.80838906 |
| 60 days | 0.56929854 | 3.07271794 | 7.42791628 | 1.9667517418919 | 1.367677165 | 4.1666824325 |
| 120 days | 0.40530824 | 2.75452268 | 4.67469834 | 1.19884652451125 | 1.602732345 | 3.1225903 |

TABLE 1b

| | Gamma tocopherol Concentration μg/mL | | | | | |
|---|---|---|---|---|---|---|
| | Male | | | | | |
| Stage | Min | Median | Max | SD | QT1 | QT3 |
| 30 days | 1.50710403 | 4.1609569 | 7.57036836 | 1.70893713676527 | 2.53020102 | 4.763287595 |
| 60 days | 0.58253435 | 2.97406469 | 5.5309079 | 1.40738354165684 | 1.506434835 | 3.90173259 |
| 120 days | 0.6828003 | 2.93657217 | 7.71758304 | 2.00541662042193 | 2.333410025 | 4.94819353 |

Statistical analysis: the results of the compositional analysis were then subject to a statistical analysis employing the following statistical model:

Gamma tocopherol=B₀+B₁age+B₂age²+B₃sex+B₄age*sex+B₅age²*sex+ε

Age is represented in both linear and quadratic terms and is measured in days. ε refers to the random effect of the model which controls for within subject variability.

The different suffixes (B₀, B₁, B₂ . . . ) represent the different estimated slopes attached to the corresponding variable (age, linear and quadratic, sex and/or their interaction).

Table II shows the estimates for timeframe differences along with the corresponding Pvalues. The results of the Statistical analysis (statistical inference) are show in table II.

TABLE II

| Timeframe | Variable | Estimate | SE | Pvalue | Unit |
|---|---|---|---|---|---|
| 30 days | Gamma | 0.06521 | 0.5055 | 0.89736 | μg/mL |
| | tocopherol | | | | |
| 60 days | Gamma | −0.19079 | 0.4900 | 0.69700 | μg/mL |
| | tocopherol | | | | |
| 90 days | Gamma | 0.12818 | 0.5436 | 0.81358 | μg/mL |
| | tocopherol | | | | |
| 120 days | Gamma | 1.02212 | 0.4814 | 0.03373 | μg/mL |
| | tocopherol | | | | |

Contrast refers to the estimated difference between male and female gamma tocopherol levels.

The estimates show that at 120 days, there is a statistically significant difference.

A P-value inferior to 0.1 for a particular timeframe suggests that there is a statistically significant difference in the gamma tocopherol content of HM produced at the specific timeframes indicated.

### Example 2

Examples of gender specific synthetic nutritional compositions (infant formulas) tailored to infants of 4 months of age or older are given in table Ill. In combination these are an example of a nutritional system of the invention.

TABLE III

| | Up to 2 months | | 2 months up to 4 | | 4 months of age | |
|---|---|---|---|---|---|---|
| | of age | | months of age | | and older | |
| | M | F | M | F | M | F |
| Ingredients | Per Liter | | Per Liter | | Per Liter | |
| Energy (kcal) | 670 | 670 | 670 | 670 | 630 | 630 |
| Protein (g) | 12.1 | 12.1 | 12.1 | 12.1 | 11.3 | 11.3 |
| Fat (g) | 35.649 | 35.649 | 35.649 | 35.649 | 31.446 | 31.458 |
| Linoleic acid (g) | 5.3 | 5.3 | 5.3 | 5.3 | 4.7 | 4.7 |
| α-Linolenic acid | 675 | 675 | 675 | 675 | 600 | 600 |
| (mg) | | | | | | |
| Lactose (g) | 74.7 | 74.7 | 74.7 | 74.7 | 75 | 75 |
| Prebiotic | 4.3 | 4.3 | 4.3 | 4.3 | 4.0 | 4.0 |
| (100% GOS) (g) | | | | | | |
| Minerals (g) | 2.5 | 2.5 | 2.5 | 2.5 | 2.3 | 2.3 |
| Na (mg) | 150 | 150 | 150 | 150 | 158 | 158 |
| K (mg) | 590 | 590 | 590 | 590 | 504 | 504 |
| Cl (mg) | 430 | 430 | 430 | 430 | 410 | 410 |
| Ca (mg) | 410 | 410 | 410 | 410 | 378 | 378 |
| P (mg) | 210 | 210 | 210 | 210 | 208 | 208 |
| Mg (mg) | 50 | 50 | 50 | 50 | 44 | 44 |
| Mn (μg) | 50 | 50 | 50 | 50 | 32 | 32 |
| Se (μg) | 13 | 13 | 13 | 13 | 19 | 19 |
| Vitamin A | 700 | 700 | 700 | 700 | 570 | 570 |
| (μg RE) | | | | | | |
| Vitamin D | 10 | 10 | 10 | 10 | 9.5 | 9.5 |
| (μg) | | | | | | |
| Vitamin E | 5.4 | 5.4 | 5.4 | 5.4 5.0 | 5.0 | 5.0 |
| (mg TE) | including | including | including | including | including | including |
| | 4 mg of | 3.5 mg of | 2.8 mg of | 3 mg of | 3.5 mg of | 2.5 mg of |
| | gamma | gamma | gamma | gamma | gamma | gamma |
| | tocopherol | tocopherol | tocopherol | tocopherol | tocopherol | tocopherol |
| Vitamin K1 | 54 | 54 | 54 | 54 | 50 | 50 |
| (μg) | | | | | | |
| Vitamin C | 67 | 67 | 67 | 67 | 95 | 95 |
| (mg) | | | | | | |
| Vitamin B1 | 0.47 | 0.47 | 0.47 | 0.47 | 0.6 | 0.6 |
| (mg) | | | | | | |
| Vitamin B2 | 1 | 1 | 1 | 1 | 0.6 | 0.6 |
| (mg) | | | | | | |
| Niacin (mg) | 6.7 | 6.7 | 6.7 | 6.7 | 3.2 | 3.2 |
| Vitamin B6 | 0.5 | 0.5 | 0.5 | 0.5 | 0.4 | 0.4 |
| (mg) | | | | | | |
| Lactoferrin | 1 | 1 | 1 | 1 | 0.3 | 0.3 |
| (bovine) g | | | | | | |
| Folic acid (μg) | 60 | 60 | 60 | 60 | 95 | 95 |
| Pantothenic | 3 | 3 | 3 | 3 | 5.0 | 5.0 |
| acid (mg) | | | | | | |
| Vitamin B12 | 2 | 2 | 2 | 2 | 1.3 | 1.3 |
| (μg) | | | | | | |
| Biotin (μg) | 15 | 15 | 15 | 15 | 12.6 | 12.6 |
| Choline (mg) | 67 | 67 | 67 | 67 | 95 | 95 |
| Fe (mg) | 8 | 8 | 8 | 8 | 6.3 | 6.3 |
| I (μg) | 100 | 100 | 100 | 100 | 95 | 95 |
| Cu (mg) | 0.4 | 0.4 | 0.4 | 0.4 | 0.4 | 0.4 |
| Zn (mg) | 5 | 5 | 5 | 5 | 5.7 | 5.7 |

### Example 3

An example of a nutritional system in accordance with the invention is given in table IV.

TABLE IV

| | Up to 4 months | 4 months of age | |
|---|---|---|---|
| | of age | and older | |
| | Gender neutral | M | F |
| Ingredients | Per Litre | Per Liter | |
| Energy (kcal) | 670 | 630 | 630 |
| Protein (g) | 12.1 | 11.3 | 11.3 |
| Fat (g) | 35.649 | 31.446 | 31.458 |
| Linoleic acid (g) | 5.3 | 4.7 | 4.7 |
| α-Linolenic acid | 675 | 600 | 600 |
| (mg) | | | |
| Lactose (g) | 74.7 | 75 | 75 |
| Prebiotic (100% | 4.3 | 4.0 | 4.0 |
| GOS) (g) | | | |
| Minerals (g) | 2.5 | 2.3 | 2.3 |
| Na (mg) | 150 | 158 | 158 |
| K (mg) | 590 | 504 | 504 |
| Cl (mg) | 430 | 410 | 410 |
| Ca (mg) | 410 | 378 | 378 |
| P (mg) | 210 | 208 | 208 |
| Mg (mg) | 50 | 44 | 44 |
| Mn (μg) | 50 | 32 | 32 |
| Se (μg) | 13 | 19 | 19 |
| Vitamin A | 700 | 570 | 570 |
| (μg RE) | | | |
| Vitamin D | 10 | 9.5 | 9.5 |
| (μg) | | | |
| Vitamin E | 5.4 | 5.0 | 5.0 |
| (mg TE) | | | |
| Vitamin K1 | 54 | 50 | 50 |
| (μg) | | | |
| Vitamin C | 67 | 95 | 95 |
| (mg) | | | |
| Vitamin B1 | 0.47 | 0.6 | 0.6 |
| (mg) | | | |
| Vitamin B2 | 1 | 0.6 | 0.6 |
| (mg) | | | |
| Niacin (mg) | 6.7 | 3.2 | 3.2 |
| Vitamin B6 | 0.5 | 0.4 | 0.4 |
| (mg) | | | |
| Lactoferrin | 1 | 0.3 | 0.3 |
| (bovine) g | | | |
| Folic acid (μg) | 60 | 95 | 95 |
| Pantothenic | 3 | 5.0 | 5.0 |
| acid (mg) | | | |
| Vitamin B12 | 2 | 1.3 | 1.3 |
| (μg) | | | |
| Biotin (μg) | 15 | 12.6 | 12.6 |
| Choline (mg) | 67 | 95 | 95 |
| Fe (mg) | 8 | 6.3 | 6.3 |
| I (μg) | 100 | 95 | 95 |
| Cu (mg) | 0.4 | 0.4 | 0.4 |
| Zn (mg) | 5 | 5.7 | 5.7 |

1. A Gender specific synthetic nutritional composition tailored for an infant comprising gamma tocopherol in a concentration reflecting the concentration found in human milk produced for an infant of the same gender and age.

2. A gender specific synthetic nutritional composition according to claim 1 wherein, the composition is tailored for an infant of an age selected from the group consisting of up to 2 months of age, 2 months up to 4 months of age, or 4 months of age or older, and wherein if the concentration of gamma tocopherol is tailored to a male infant of up to 2 months of age it is within the range of 1.50 to 7.57 μg/mL and, if the concentration of gamma tocopherol is tailored to a female infant of up to 2 months of age it is within the range of 1.17 to 8.18 μg/mL; if the concentration of gamma tocopherol is tailored to a male infant of 2 months to 4 months of age it is within the range of 0.58 to 5.3 μg/mL and, if the concentration of gamma tocopherol is tailored to a female infant of 2 months to 4 months of age it is within the range of 0.57 to 7.42 μg/mL; and if the concentration of gamma tocopherol is tailored to a male infant of 4 months of age or older it is within the range of 0.68 to 7.727 μg/mL and, if the concentration of gamma tocopherol is tailored to a female infant of 4 months of age or older it is within the range of 0.4 to 4.7 μg/mL.

3. A gender specific synthetic nutritional composition according to claim 1 wherein, the gender specific synthetic nutritional composition is selected from the group consisting of: infant formula, and a composition for infants that is intended to be added to or diluted with human milk.

4-10. (canceled)

11. A method for providing an optimum amount of gamma tocopherol to an infant comprising:
a. preparing a gender specific synthetic nutritional composition comprising gamma tocopherol in a concentration reflecting the concentration found in human milk produced for an infant of the same gender and age from a gender neutral synthetic nutritional composition; and
b. feeding the gender specific synthetic nutritional composition to an infant.

12. (canceled)

13. A kit for providing an optimized amount of gamma tocopherol to an infant, the kit comprising:
a. A gender neutral synthetic nutritional composition
b. A label indicating dosage requirements for an infant so as to arrive at a gender specific synthetic nutritional composition comprising gamma tocopherol in a concentration reflecting the concentration found in human milk produced for an infant of the same gender and age.
